(19) United States
(12) Patent Application Publication
(10) Pub. No.: US 2004/0143218 A1
(43) Pub. Date: Jul. 22, 2004

(54) Needle having optimum grind for reduced insertion force

(75) Inventor: Kusal Das, Wrightstown, PA (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Animas Corporation

(21) Appl. No.: 10/347,985

(22) Filed: Jan. 21, 2003

(51) Int. Cl. A61M 5/178

(52) U.S. Cl. 604/164.06

(57) ABSTRACT

An introducer needle for an infusion set or the like includes a solid tip portion with a plurality of intersecting wedge surfaces that converge toward a common insertion point. Preferably, the common insertion point is coincident with a central axis of the introducer needle. The tip portion also includes a plurality of cutting edges formed at intersections of the wedge surfaces. The cutting edges converge toward the common insertion point so that insertion of the introducer needle into the membrane causes even cutting and separation of the membrane around the introducer needle.

### BACKGROUND OF THE INVENTION

This invention relates to medical devices, and more particularly to an introducer needle arranged to facilitate membrane penetration and installation of a cannula or catheter within the membrane.

The delivery of medication or other fluids is often accomplished through a cannula or catheter that is typically placed either subcutaneously or intravenously. For subcutaneous delivery of fluids, the use of an infusion set or injection port reduces the need to constantly puncture the skin and provides a method of temporarily detaching the fluid line for activities such as dressing or bathing. Infusion sets typically include a cannula and an introducer needle that extends through the cannula. A self-adhesive pad is often used to secure the cannula against movement once installed. A manual inserter is often used to install the fusion set. The manual inserter typically includes a housing and a spring-loaded sliding mechanism located in the housing to which the infusion set is temporarily connected. In use, the user typically grasps the inserter housing with one hand while pinching a fold of skin between the thumb and forefinger of the other hand. The sliding mechanism is then released to force the introducer needle and the outer end of the cannula into the fold of skin. The introducer needle is then removed, leaving the cannula installed in the subcutaneous layer. The cannula can then be secured against movement. A tubing from a fluid supply source, such as an insulin pump, can be connected to the cannula housing to deliver insulin or other substances to the subcutaneous layer through the cannula.

The introducer needle is typically constructed of a hollow, metallic tube with one end ground at an angle to form a cutting tip that is offset from a central axis of the tube. Insertion of the introducer needle into the skin typically requires a relatively high insertion force, due at least in part to the offset nature of the cutting tip. The combination of relatively high insertion force and the offset nature of the tip may create a bending moment about the introducer needle during insertion. When the bending moment is relieved, such as when the introducer needle pierces the skin, the surrounding tissue may be damaged and discomfort may be increased as the introducer needle attempts to spring back to its original shape.

In addition, burr formation is often prevalent during manufacture of the angle ground hollow introducer needle. The removal of such burrs can be time consuming and difficult.

Furthermore, during assembly of an infusion set, the offset cutting tip of the introducer needle can contact and damage the inner side wall of the cannula. Thus, great care is needed to ensure that the introducer needle and cannula are properly aligned during insertion of the introducer needle through the cannula.

### BRIEF SUMMARY OF THE INVENTION

According to one aspect of the invention, an introducer needle for penetrating a membrane comprises a solid body portion having a central axis and a tip portion extending from the solid body portion. The tip portion includes a plurality of intersecting wedge surfaces that converge toward a common insertion point from the solid body portion. Preferably, the common insertion point is coincident with the central axis of the solid body portion. The tip portion also includes a plurality of cutting edges formed at intersections of the wedge surfaces. The cutting edges converge toward the common insertion point from the solid body portion. With this arrangement, insertion of the introducer needle into the membrane causes even cutting and separation of the membrane around the introducer needle.

According to a further aspect of the invention, an infusion set comprises a cannula housing, a cannula extending from the housing, and an introducer needle extending through at least a portion of the cannula. The introducer needle comprises a solid body portion having a central axis and a tip portion extending from the solid body portion. The tip portion has a plurality of intersecting wedge surfaces that converge toward a common insertion point from the solid body portion and a plurality of cutting edges formed at intersections of the wedge surfaces, with the cutting edges converging toward the common insertion point from the solid body portion. Preferably, the common insertion point is coincident with the central axis of the solid body portion.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a front perspective view of an inserter assembly incorporating an infusion set with an introducer needle in accordance with the present invention;

FIG. 2 is an enlarged perspective view of a portion of the introducer needle and cannula;

FIG. 3 is a side elevational view of a portion of the introducer needle;

FIG. 4 is a front elevational view of the introducer needle; and

FIG. 5 is a front elevational view of an introducer needle in accordance with a further embodiment of the invention.

It is noted that the drawings are intended to represent only typical embodiments of the invention and therefore should not be construed as limiting the scope thereof. The invention will now be described in greater detail with reference to the drawings, wherein like parts throughout the drawing figures are represented by like numerals.

### DETAILED DESCRIPTION OF THE INVENTION

Certain terminology may be used in the following description for convenience only and is not limiting. The words “left,” “right,” “upper,” and “lower” designate directions in the drawings to which reference is made. The words “inwardly” and “outwardly” refer to directions toward and away from, respectively, the geometric center of the needle and designated parts thereof. The terminology includes the words above specifically mentioned, derivatives thereof, and words of similar import.

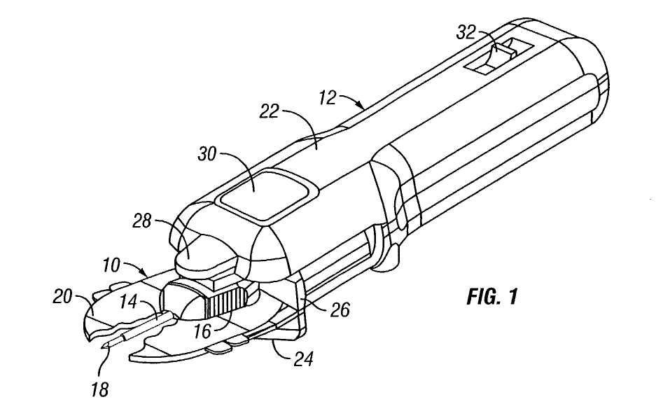

Referring now to the drawings, and to FIG. 1 in particular, an infusion set 10 in accordance with the present invention is shown temporarily positioned on an inserter assembly 12 for installation in the subcutaneous skin layer of a user.

The infusion set 10 includes a cannula 14 extending from a generally hollow cannula housing 16 and an introducer needle 18 extending through the cannula 14 and into the cannula housing 16. The cannula 14 is preferably constructed of a fluoropolymer material, such as Teflon™, or other inert material. An adhesive-backed pad 20 is preferably attached to the cannula housing 16 with the adhesive layer (not shown) facing away from the cannula housing 16. A portion of the pad 20 is shown broken away in FIG. 1 to more clearly illustrate the introducer needle 18 and cannula 14 extending underneath the housing. It will be understood that the infusion set 10 is not limited to the low-profile type, but may be arranged to insert the introducer needle 18 and cannula 14 at different angles, including perpendicular, to the skin surface.

The inserter 12 includes an inserter housing 22 with an angled alignment guide 24 formed therewith so that the infusion set 10 can be inserted into the skin at a predetermined angle. The inserter 12 also includes a spring-loaded slide assembly 26 that is biased toward a forward position as shown, and is retractable to a rearward or cocked position. A locking mechanism 28 is adapted to hold the slide assembly 26 in the cocked position, and an actuating button 30 is operatively associated with the locking mechanism 28 for releasing the slide assembly 26. A locking lever 32 can also be provided for preventing inadvertent depression of the actuating button 30, and thus inadvertent release of the slide assembly 26. Further details of the inserter assembly can be found in copending U.S. patent application Publication No. 2002/0077599 filed on Dec. 18, 2001, the disclosure of which is hereby incorporated by reference. It will be understood that the infusion set 10 can be used with inserters of different configurations, or can be used without an inserter.

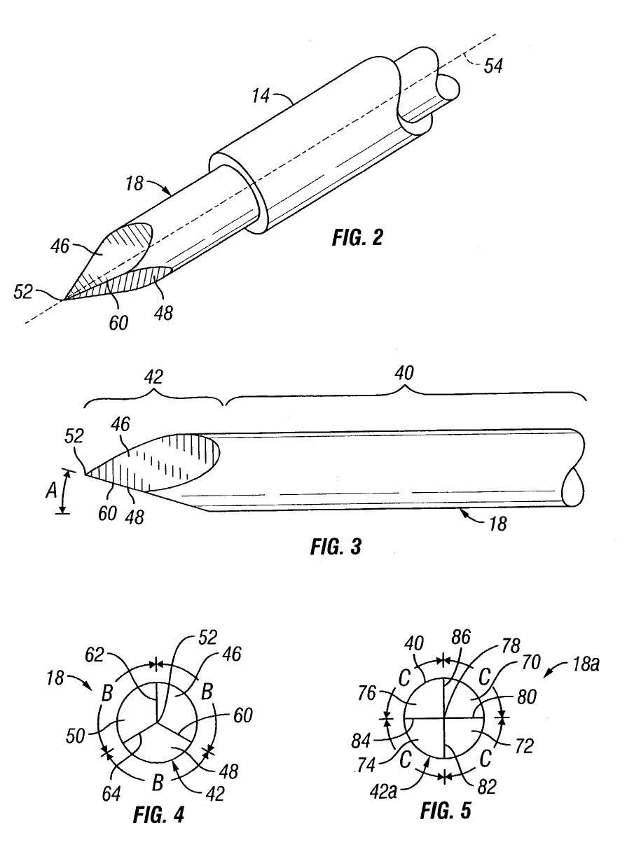

With reference now to FIGS. 2-4, the introducer needle 18 includes a body portion 40 and a tip portion 42 that is integrally formed with, and extends from, the body portion 40. Preferably, the body portion 40 and tip portion 42 are of solid construction. As shown, the body portion 40 is circular in cross section, but may be configured with other cross sectional shapes, such as triangular, square, oval, and so on.

The tip portion 42 preferably includes a plurality of substantially planar wedge faces, such as first wedge face 46, second wedge face 48 and third wedge face 50 that converge toward a common insertion point 52 from the body portion 40. The common insertion point 52 is preferably coincident with a central axis 54 of the introducer needle 18. Preferably, the wedge faces extend from the solid body portion 40 to the central axis 54 at an angle A in the range of approximately 5-30 degrees, and more preferably at an angle A of approximately 15 degrees. As shown, the first and second wedge faces 46 and 48 intersect to form a first cutting edge 60, the first and third wedge faces 46 and 50 intersect to form a second cutting edge 62, and the second and third wedge faces 48 and 50 intersect to form a third cutting edge 64. The cutting edges 60, 62 and 64 preferably converge toward the common insertion point 52 from the solid body portion 40 and are preferably circumferentially spaced about the central axis 54 at an angle B of approximately 120 degrees. Since the cutting edges extend along the intersection of the wedge faces, they also extend or slope from the solid body portion 40 to the common insertion point or central axis 54 at an angle A in the range of approximately 5-30 degrees, and more preferably at an angle A of approximately 15 degrees. In this manner, the length of each cutting edge 60, 62 and 64 can be less than a cutting length of the prior art angle ground hollow needle to thereby reduce material costs.

The introducer needle 18 is preferably constructed from a solid rod or bar of material, such as stainless steel, by cutting the rod or bar to a predetermined length, then grinding or otherwise forming one end of the rod or bar to form the tip portion 42.

Referring again to FIG. 1, in use, and by way of example, the infusion set 10 is loaded onto the slide assembly 26 of the inserter assembly 12. The slide assembly 26 is then retracted to the cocked position and held in place by the locking mechanism 28. The inserter assembly 12 is then positioned against the skin of a user and the button 30 is depressed to release the slide assembly 26. The infusion set 10 moves together with the slide assembly under spring bias toward the skin until the introducer needle 18 and cannula 14 are positioned in the subcutaneous layer. The concentric location of the insertion point 52 together with the symmetrically spaced cutting edges 60, 62 and 64 facilitate insertion of the introducer needle 18 into the skin with a concentric coaxial force. This coaxial force has no bending component, as in the prior art angle ground hollow needles. The three cutting edges 60, 62 and 64 cooperate with the concentric insertion point 52 and the wedge faces 46, 48 and 50 to minimize skin displacement during needle insertion while assuring a clean cut through the tissue. In addition, the solid construction of the introducer needle 18 gives added strength over prior art hollow body constructions.

Still another advantage of the above-described introducer needle 18 is less burr formation and easier burr removal during manufacturing than in the prior art angle grinding of hollow needles.

Still further, the introducer needle 18 facilitates assembly of the infusion set 10. When inserted through the cannula 14 during assembly, the introducer needle 18 glides through without touching or damaging the cannula 14 since the insertion point 52 is spaced away from the wall of the cannula.

Referring now to FIG. 5, a front elevational view of an introducer needle 18a in accordance with a further embodiment of the invention is illustrated. The introducer needle 18a has a tip portion 42a with a plurality of substantially planar wedge faces, such as first wedge face 70, second wedge face 72, third wedge face 74, and fourth wedge face 76 that converge toward a common insertion point 78 from the body portion 40. The insertion point 78 is preferably coincident with a central axis of the introducer needle 18A. Preferably, the wedge faces extend from the solid body portion 40 to the central axis 78 at an angle A in the range of approximately 5-30 degrees, and more preferably at an angle A of approximately 15 degrees. As shown, the first and second wedge faces 70 and 72 intersect to form a first cutting edge 80, the second and third wedge faces 72 and 74 intersect to form a second cutting edge 82, the third and fourth wedge faces 74 and 76 intersect to form a third cutting edge 84, and the fourth and first wedge faces 76 and 70 intersect to form a fourth cutting edge 86. The cutting edges 80-86 preferably converge toward the insertion point 78 from the solid body portion 40 and are preferably circumferentially spaced about the central axis 54 at an angle C of approximately 90 degrees. Since the cutting edges extend along the intersection of the wedge faces, they also extend or slope from the solid body portion 40 to the central axis 78 at an angle A that is preferably in the range of approximately 5-30 degrees, and more preferably at an angle A of approximately 15 degrees.

While the two embodiments of the invention shown in FIGS. 4 and 5 illustrate inserter needles with 3 and 4 edge faces, it should be understood that the invention is not so limited and that 5 or more faces can be used.

While the invention has been taught with specific reference to the above-described embodiments, those skilled in the art will recognize that changes can be made in form and detail without departing from the spirit and the scope of the invention. By way of example, it will be understood that the invention is not limited to the particular number of wedge faces and cutting edges illustrated, but may have more or less faces and cutting edges. In addition, the angles between the cutting edges and/or their slopes can be asymmetrical. Moreover, although the introducer needle has been taught for use with an inserter assembly, it will be understood that the introducer needle is not so limited, but may be used in other devices and/or methods for positioning a cannula or catheter in a membrane or tissue and/or for creating a passage or opening in a membrane or tissue. Thus, the described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

I/we claim:

1. An introducer needle for penetrating a membrane, the needle comprising:
a solid body portion having a central axis; and
a solid tip portion extending from the solid body portion, the solid tip portion comprising:
a common insertion point that is coincident with the central axis;
a plurality of wedge surfaces extending from the solid body portion and converging toward the common insertion point, each wedge surface forming an intersection with an adjacent wedge surface; and
a plurality of cutting edges formed at the intersections of the wedge surfaces, the cutting edges converging toward the common insertion point;
wherein insertion of the introducer needle into the membrane causes even cutting and separation of the membrane around the introducer needle.

2. An introducer needle in accordance with claim 1, wherein the plurality of wedge surfaces comprises first, second and third wedge surfaces, with the first and second wedge surfaces intersecting at a first intersection, the first and third wedge surfaces intersecting at a second intersection, and the second and third wedge surfaces intersecting at a third intersection.

3. An introducer needle in accordance with claim 2, wherein a first angle is formed between the first and second wedge surfaces, a second angle is formed between the first and third wedge surfaces, and a third angle is formed between the third and first wedge surfaces.

4. An introducer needle in accordance with claim 2, wherein the plurality of cutting edges comprises a first cutting edge located at the first intersection, a second cutting edge located at the second intersection, and a third cutting edge located at the third intersection.

5. An introducer needle in accordance with claim 4, wherein the first, second and third angles are each approximately 120 degrees

6. An introducer needle in accordance with claim 5, wherein the first, second and third cutting edges have a slope in the range of about 5 degrees to about 30 degrees with respect to the central axis.

7. An introducer needle in accordance with claim 6, wherein the first, second and third cutting edges have a slope of about 15 degrees with respect to the central axis.

8. An introducer needle in accordance with claim 4, wherein the first, second and third cutting edges have a slope in the range of about 5 degrees to about 30 degrees with respect to the central axis

9. An introducer needle in accordance with claim 1, wherein each wedge surface extends at an angle in the range of about 5 degrees to about 30 degrees with respect to the central axis.

10. An introducer needle in accordance with claim 9, wherein each wedge surface extends at an angle of about 15 degrees with respect to the central axis.

11. An introducer needle in accordance with claim 1, wherein each wedge surface is planar.

12. An infusion set comprising:
a cannula housing;
a cannula extending from the cannula housing; and
an introducer needle extending through at least a portion of the cannula, the introducer needle comprising:
a solid body portion having a central axis;
a solid tip portion extending from the solid body portion, the solid tip portion comprising:
a common insertion point that is coincident with the central axis;
a plurality of wedge surfaces extending from the solid body portion and converging toward the common insertion point, each wedge surface forming an intersection with an adjacent wedge surface; and
a plurality of cutting edges formed at the intersections of the wedge surfaces, the cutting edges converging toward the common insertion point;
wherein insertion of the introducer needle into the membrane causes even cutting and separation of the membrane around the introducer needle.

13. An infusion set in accordance with claim 12, wherein the plurality of wedge surfaces comprises first, second and third wedge surfaces, with the first and second wedge surfaces intersecting at a first intersection, the first and third wedge surfaces intersecting at a second intersection, and the second and third wedge surfaces intersecting at a third intersection.

14. An infusion set in accordance with claim 13, wherein a first angle is formed between the first and second wedge surfaces, a second angle is formed between the first and third wedge surfaces, and a third angle is formed between the third and first wedge surfaces.

15. An infusion set in accordance with claim 14, wherein the plurality of cutting edges comprises a first cutting edge located at the first intersection, a second cutting edge located at the second intersection, and a third cutting edge located at the third intersection.

16. An infusion set in accordance with claim 15, wherein the first, second and third angles are each approximately 120 degrees

17. An infusion set in accordance with claim 16, wherein the first, second and third cutting edges have a slope in the range of about 5 degrees to about 30 degrees with respect to the central axis.

18. An infusion set in accordance with claim 17, wherein the first, second and third cutting edges have a slope of about 15 degrees with respect to the central axis.

19. An infusion set in accordance with claim 15, wherein the first, second and third cutting edges have a slope in the range of about 5 degrees to about 30 degrees with respect to the central axis

20. An infusion set in accordance with claim 12, wherein each wedge surface extends at an angle in the range of about 5 degrees to about 30 degrees with respect to the central axis.

21. An infusion set in accordance with claim 20, wherein each wedge surface extends at an angle of about 15 degrees with respect to the central axis.

22. An infusion set in accordance with claim 12, and further comprising a cannula housing, with the cannula and introducer needle extending from the cannula housing.

23. An infusion set in accordance with claim 22, and further comprising an adhesive pad connected to the cannula housing for securing the cannula housing to the skin of a user.

24. An infusion set in accordance with claim 12, wherein each wedge surface is planar.
